(19) United States
(12) Patent Application Publication
(10) Pub. No.: US 2019/0046637 A1
(43) Pub. Date: Feb. 14, 2019

(54) MATERIALS AND METHODS FOR MITIGATING IMMUNE-SENSITIZATION

(71) Applicant: BIOSTRATEGIES LC, State University, AR (US)

(72) Inventors: DAVID N. RADIN, Jonesboro, AR (US); WALTER ACOSTA, Jonesboro, AR (US)

(21) Appl. No.: 16/079,932

(22) PCT Filed: Mar. 1, 2017
(86) PCT No.: PCT/US2017/020235

§ 371 Date: Aug. 24, 2018

Related U.S. Application Data
(60) Provisional application No. 62/301,973, filed on Mar. 1, 2016.
(60) Provisional application No. 62/456,443, filed on Feb. 8, 2017.

(51) Int. Cl. A61K 39/39; A61K 38/16; A61K 38/47; A61P 3/00

(52) U.S. Cl. CPC A61K 39/39; A61K 38/168; A61K 2039/55516; C12Y 302/01076; A61P 3/00; A61K 38/47

(57) ABSTRACT

The present invention concerns materials and method for delivery and internalization of an agent into a cell even in the presence of an immune response, such as antibodies or antisera or immune cells that bind to the agent. The agent can be a compound, drug, peptide, protein, nucleic acid, antigen, immunogen, or other biological molecule. In one embodiment, the agent is operatively linked to a lectin-based carrier. The present invention can be used for delivery and cellular internalization of any entity where an immune response to the entity is present or is likely to be produced or developed. The present invention also concerns methods and materials for providing for an adjuvant and carrier for vaccinations of a person or animal. The present invention also concerns a method for treating or preventing a disease or condition in a human or animal wherein the human or animal has produced or will produce an immune response against a therapeutic agent that can treat said disease or condition, the method comprising administering to the human or animal an effective amount of said therapeutic agent operatively linked to a lectin-based carrier.

### CROSS-REFERENCE TO RELATED APPLICATIONS

The present application claims the benefit of U.S. Provisional Application Ser. No. 62/301,973, filed Mar. 1, 2016, and 62/456,443, filed Feb. 8, 2017, each of which is hereby incorporated by reference herein in its entirety, including any figures, tables, nucleic acid sequences, amino acid sequences, or drawings.

### BACKGROUND OF THE INVENTION

Enzyme replacement therapies (ERTs) remain the most effective treatment for those rare genetic diseases for which approved recombinant enzymes are available. ERTs have been crucial in treating several lysosomal storage diseases (LSD), which in their severe forms present with devastating multi-organ pathologies in affected children. However, the induction of patient antibodies (immune sensitization) directed against the therapeutic enzyme has emerged as a significant limitation in the effectiveness of ERTs, altering enzyme distribution and activity. Reported rates of patients that develop significant immune sensitization responses following ERT are 91%, 97% and 100% for Hurler (Mucopolysaccharidosis I; MPS I), Maroteaux-Lamy (MPS VI), and Pompe diseases, respectively (Dickson et al., 2008). Because early/infantile-onset forms comprise the most severe LSD mutations (“nulls” with little/no detectable enzyme), the development of immune sensitization is much more prevalent in young patients. These children can show dramatic, often life-saving, improvements upon treatment onset. However, progress quickly declines as these children develop neutralizing antibodies to the therapeutic enzyme.

The effectiveness of enzyme replacement therapies (ERT) for rare genetic diseases and other peptide-, protein-, or glycoprotein-based therapies can be undermined by the development of anti-drug antibodies (ADA), also termed immune-sensitization. All currently approved ERTs for LSDs, with the exception of ERTs for Gaucher Disease, exploit the mannose-6-phosphate (M6P) receptor for uptake into cells. Anti-ERT antibodies that interfere with M6P-mediated uptake constitute the predominant “neutralizing” class of antibodies (Glaros et al., 2002).

### BRIEF SUMMARY OF THE INVENTION

The present invention concerns materials and method for delivery and internalization of an agent into a cell even in the presence of an immune response, such as antibodies or antisera, that binds to the agent. The agent can be a compound, drug, peptide, protein, nucleic acid, antigen, immunogen, or other biological molecule. In the method of the invention, the agent is operatively linked to a lectin-based carrier. The present invention can be used for delivery and cellular internalization of any entity where an immune response to the entity is present or is likely to be produced or developed. The present invention also concerns methods and materials for providing for an adjuvant and/or carrier for vaccinations or immunizations of a person or animal wherein immune responses are induced to the antigen or immunogen but only minimally to the lectin-based carrier. The present invention also concerns a method for treating a disease or condition in a human or animal wherein the human or animal has produced or will produce an immune response against a therapeutic agent that can treat said disease or condition, the method comprising administering to the human or animal an effective amount of the therapeutic agent operatively linked to a lectin-based carrier.

The invention exploits the unexpected low immunogenicity of the lectin-based carrier compared to the associated therapeutic and/or antigenic agent, which enables appropriate distribution and efficacy of agent in individuals in which anti-agent immune responses reduce desired therapeutic or immunogenic responses.

### BRIEF DESCRIPTION OF THE DRAWINGS

FIG. 1. IDUA:RTB uptake in presence of inhibitory levels of anti-IDUA antibodies. IDUA:RTB and mammalian cell-derived IDUA (rhIDU) were pre-incubated with canine sera from dogs no longer responsive to rhIDU therapy (gift of P. Dickson, UCLA) were used to treat MSP I fibroblasts. Intracellular IDUA activity was analyzed at 1 and 4 h and compared to fibroblasts treated in the absence of neutralizing sera (Acosta, Cramer unpub.).

FIGS. 2A and 2B. ERT blocking antibodies. From Ponder, 2008 (Ponder, 2008).

FIG. 3. RTB trafficking.

FIG. 4. RTB:IDUA corrects GAG levels+/−MMR and M6PR inhibitors. GAG levels were measured in normal (GM00010) or MPS-I (GM01391) fibroblasts that were untreated or treated for 24 hr with 1 unit IDUA eq./ml using RTB:IDUA or mcd-IDUA (mammalian cell-derived IDUA). For inhibitor studies, MPS-I cells were pre-incubated for 2 hr with 4 mM M6P or 4 mg/ml mannan prior to IDUA treatment (Acosta et al., 2016).

FIGS. 5A and 5B. The RTB carrier does not elicit antibody responses in mice receiving multiple administrations. MPS I mice were treated with 8 weekly injections of IDUA:RTB at 0.58 mg/kg (human IDUA dose) or 2.0 mg/kg. Sera, collected at 14, 35, and 63 days after initial treatment, was analyzed for presence of anti-IDUA (FIG. 5A) and anti-RTB (FIG. 5B) IgGs. Data presented is relative titer levels (anti-IgG ODs) at 63 d after 8 treatments; n=8 for treated cohorts; n=6 for untreated mice. Both IDUA:RTB treatment groups showed significant mitigation of disease symptom.

FIG. 6. Antibody isotyping in terminal serum after 8 weekly administration of 0.58 mg/Kg or 2.0 mg/Kg of IDUAL to MPS I −/− mice

FIG. 7. GFP-specific serum IgG responses in ICR mice following intranasal immunization with

1) 0.1 μg GFP

2) 0.1 μg GFP+0.1 μg cholera toxin (CT) adjuvant

3) 0.1 μg GFP+1 μg cholera toxin (CT)

4) 0.1 μg GFP+control non-transgenic hairy root media

5) 0.1 μg GFP as a RTB:GFP fusion purified from transgenic hairy root media.

Groups of 5 mice were immunized, boosted on a 2-week schedule, and bled 6 days after each boost. Titers were determined by ELISA and were defined as the reciprocal of the highest dilution of the serum giving an absorbance of ≥0.2 (three replicates per determination). Each value is average+standard error of each group. (Medina-Bolivar et al., 2003).

FIG. 8. Adjuvant-specific serum IgG responses (anti-RTB or anti-CT) in mice trial described in FIG. 7 following nasal immunization with:

1) 0.1 μg GFP fused to 0.12 μg RTB

2) 0.1 μg GFP+0.1 μg CT

3) 0.1 μg GFP+1.0 μg CT.

FIG. 9. Aim 2 overview and workflow.

FIG. 10. A timeline for immunization and sample collection.

FIGS. 11A and 11B. IDUA:RTB treatment of MPS-I mice. FIG. 11A. IDUA activity in untreated mice or mice 24 hr after IDUA:RTB injection. FIG. 11B. GAG levels 5 dpi. (Acosta et al., 2016; Ou et al., 2016).

### BRIEF DESCRIPTION OF THE SEQUENCES

SEQ ID NO:1 is the amino acid sequence of a modified patatin sequence that can be used in the present invention.

### DETAILED DESCRIPTION OF THE INVENTION

The present invention encompasses the use of plant lectin-based delivery of associated bioactive molecules (e.g., drugs) whereby the lectin-delivery-module enables efficacious drug delivery even in the presence of anti-drug antibodies to the bioactive molecule. This inventions provides unique advantages in drug delivery in ADA immune-sensitized patients independent of whether the elicitation of immunogenicity was initiated by
Multiple administrations of bioactive component such as an ERT that lacks our inventive lectin carrier; i.e., patients previously treated with a drug that subsequently developed ADA that compromises drug treatment effectiveness
Treatment with the drug operatively associated with the lectin-delivery-module resulting in antibodies raised to the bioactive drug component (for example, as lectin-replacement enzyme fusion)
Treatment by DNA- or RNA-based gene therapy approaches that direct synthesis of the bioactive molecule in vivo leading to ADA directed against the encoded therapeutic product.

These applications for patient treatments are based on the surprising discovery that 1) the plant lectin B-subunit of ricin (RTB) is able to carry its drug cargo into cells and direct appropriate subcellular trafficking and transcytosis even in the presence of inhibitory levels of neutralizing antibodies directed against its cargo, 2) the delivered cargo shows bioactivity upon target-site delivery, and 3) the RTB lectin carrier itself shows surprisingly low immunogeniticy and does not suffer from reduced efficacy due to anti-carrier antibodies. The RTB carrier mediates delivery and effective biodistribution of corrective ERT enzyme in animals that are immune sensitized to a model ERT product. The invention provides for broad application of the lectin-delivery platform to treatments for lysosomal diseases, other genetic disorders, or drug delivery in any case where immune sensitization to the drug is problematic for therapeutic outcomes. Its utility is both for patients for whom their current treatment has declined in efficacy due to ADA and for naive patients for initiating and maintaining treatment with the enzyme-lectin fusion providing long-term sustainable treatment that does not become compromised by ADA. Thus, the present invention brings new immune-mitigating ERTs to patients that provide sustainable efficacy for ERT treatments of lysosomal storage diseases and other genetic diseases.

One aspect of the present invention concerns materials and method for delivery and internalization of an agent into a cell even in the presence of an immune response, such as antibodies, antisera, and/or immune cells (e.g., B cells, T cells, etc.), that binds to the agent. In one embodiment, the antibodies, antisera, and/or immune cells are neutralizing antibodies, antisera, and/or immune cells. In the methods of the invention, the agent is operatively linked to a lectin-based carrier (LBC). The agent can be any compound, drug, peptide, protein, nucleic acid, antigen, immunogen, or other synthetic or biological molecule. In a specific embodiment, the lectin-based carrier is a non-toxic carbohydrate binding subunit of a plant lectin. In an exemplified embodiment, the plant lectin is the B-subunit of ricin (RTB), or a functional fragment or variant thereof. In specific embodiments, the ricin B subunit that is utilized is truncated by removal of about 1 to 10 amino acids at the N-terminus of the protein. In a further embodiment, the ricin B subunit is truncated wherein the first six amino acids of the protein are removed. In another embodiment, the lectin is the nigrin B B-subunit (NBB) from Sambucus nigra, or a functional fragment or variant thereof. In one embodiment, the agent is α-L-iduronidase, or an enzymatically active fragment or variant thereof. The present invention can be used for delivery and cellular internalization of any entity where an immune response or immune components to the entity are present or are likely to be produced or developed.

The present invention also concerns a method for treating or preventing a disease or condition in a human or animal wherein the human or animal has produced or will produce an immune response against a therapeutic agent that can treat said disease or condition, the method comprising administering to the human or animal an effective amount of the therapeutic agent operatively linked to a lectin-based carrier. The immune response can be an antibody response and/or an immune cell response. In one embodiment, the antibody and/or immune cells are neutralizing antibody and/or immune cells. The agent can be any compound, drug, peptide, protein, nucleic acid, antigen, immunogen, or other synthetic or biological molecule that is utilized in treating or preventing a disease or condition. In a specific embodiment, the lectin-based carrier is a non-toxic carbohydrate binding subunit of a plant lectin. In a further embodiment, the plant lectin is the B-subunit of ricin (RTB), or a functional fragment or variant thereof. In specific embodiments, the ricin B subunit that is utilized is truncated by removal of about 1 to 10 amino acids at the N-terminus of the protein. In an exemplified embodiment, the ricin B subunit is truncated wherein the first six amino acids of the protein are removed. In another embodiment, the lectin is the nigrin B B-subunit (NBB) from Sambucus nigra, or a functional fragment or variant thereof.

The subject invention concerns materials and methods for the delivery of therapeutic agents, such as drugs, peptides, proteins, antigens, immunogens, and polynucleotides, to a person or animal that has or will develop an immune response, such as an antibody response and/or immune cells response, against the therapeutic agent. In one embodiment, the antibody and/or immune cells are neutralizing antibody and/or immune cells. A method of the invention comprises administering a lectin that comprises a therapeutic agent to a person or animal in need of the therapeutic agent. In one embodiment, the person or animal has already developed antibodies against the therapeutic agent prior to administration. In another embodiment, the person or animal is at risk of developing antibodies against the therapeutic agent. Any suitable lectin, such as a plant lectin, is contemplated for use in the method. In a specific embodiment, the lectin-based carrier is a non-toxic carbohydrate binding subunit of a plant lectin. In a further embodiment, the plant lectin is the B-subunit of ricin (RTB), or a functional fragment or variant thereof. In specific embodiments, the ricin B subunit that is utilized is truncated by removal of about 1 to 10 amino acids at the N-terminus of the protein. In an exemplified embodiment, the ricin B subunit is truncated wherein the first six amino acids of the protein are removed. In another embodiment, the lectin is the nigrin B B-subunit (NBB) from Sambucus nigra, or a functional fragment or variant thereof.

In some embodiments of the present invention, the therapeutic agent is fused or linked to the subunit B, or a fragment or variant thereof, of an AB toxin. In some embodiments, the subunit B lectin protein is from ricin. In some embodiments, the ricin B subunit that is utilized is truncated by removal of about 1 to 10 amino acids at the N-terminus of the protein. In one embodiment, the ricin B subunit is truncated wherein the first six amino acids of the protein are removed. A fusion protein (or other compound) may be produced by construction of a fusion gene incorporating a nucleotide sequence encoding a lectin (such as the subunit B lectin) and a nucleotide sequence encoding the therapeutic protein, and introducing this new genetic fusion (fusion gene) into a protein expression system, expressing the fusion protein encoded by the fusion gene, and isolating the fused protein for use as a therapeutic drug. Alternatively, the fusion may be accomplished by direct chemical fusion or conjugation yielding fusion of the lectin (such as a subunit B protein) with the therapeutic agent. In one embodiment, the fusion protein comprises a linker or spacer sequence of amino acids between the lectin and the therapeutic protein or compound. Examples of linker or spacer sequences are well known in the art. Methods for preparing fusion genes and fusion protein are also well known in the art and have been described, for example, in U.S. Pat. Nos. 7,964,377; 7,867,972; 7,410,779; 7,011,972; 6,884,419; and 5,705,484.

The present invention also concerns methods and materials for providing for an adjuvant and carrier for immunizations or vaccinations of a person or animal wherein immune responses are induced to the antigen or immunogen but only minimally to the lectin-based carrier. This advantageously allows for multiple vaccinations and/or boosters, and reuse of the lectin-based carrier for multiple vaccine targets. In one method of the invention, a person or animal is administered an effective amount of an antigen or immunogen, wherein the antigen or immunogen is provided operatively linked to a lectin-based carrier (LBC). The antigen:LBC or immunogen:LBC is administered to a person or animal to generate an immune response against the antigen or immunogen. In one embodiment, the antigen:LBC or immunogen:LBC is administered to the person or animal multiple times over a period of time. In a specific embodiment, the LBC is a non-toxic carbohydrate binding subunit of a plant lectin. In an exemplified embodiment, the plant lectin is the B-subunit of ricin (RTB), or a functional fragment or variant thereof. In specific embodiments, the ricin B subunit that is utilized is truncated by removal of about 1 to 10 amino acids at the N-terminus of the protein. In a further embodiment, the ricin B subunit is truncated wherein the first six amino acids of the protein are removed. In another embodiment, the lectin is the nigrin B B-subunit (NBB) from Sambucus nigra, or a functional fragment or variant thereof. The present invention can be used as a vaccine delivery system for any known immunogen or vaccine or for any immunogen or vaccine developed in the future. In one embodiment, the immune response generated using the present invention comprises the production of antibodies that bind to one or more epitopes of the antigen or immunogen. In a further embodiment, the immune response generated is primarily a Th₂ response. In an exemplified embodiment, the antigen:LBC or immunogen:LBC is administered at a mucosal location of the person or animal, e.g., nasal administration. Compositions of the antigen:LBC or immunogen:LBC can optionally comprise other adjuvants known in the art (e.g., alum, Freund's adjuvant, etc.) and/or physiologically-acceptable buffers, etc.

Plant lectins that are contemplated within the scope of the invention include, but are not limited to those B subunits from AB toxins such as ricins, abrins, nigrins, and mistletoe toxins, viscumin toxins, ebulins, pharatoxin, hurin, phasin, and pulchellin. They may also include lectins such as wheat germ agglutinin, peanut agglutinin, and tomato lectin that, while not part of the AB toxin class, are still capable of binding to animal cell surfaces and mediating endocytosis and transcytosis. Specific examples of plant lectins including their binding affinities and trafficking behavior are discussed further below. Therapeutic compounds and agents contemplated within the scope of the invention include, but are not limited to large molecular weight molecules including therapeutic proteins and peptides, siRNA, antisense oligonucleotides, and oligosaccharides. Other therapeutic compounds and agents contemplated within the scope of the invention include small molecular weight drug compounds including but not limited to vitamins, co-factors, effector molecules, and inducers of health promoting reactions.

Additional plant lectins that are contemplated within the scope of the invention are those having particular carbohydrate binding affinities including but not limited to lectins that bind glucose, glucosamine, galactose, galactosamine, N-acetyl-glucosamine, N-acetyl-galactosamine, mannose, fucose, sialic acid, neuraminic acid, and/or N-acetylneuraminic acid, or have high affinity for certain target tissue or cells of interest. There are hundreds of plant lectins that have been identified and experimental strategies to identify plant lectins, their respective genes, and their sugar binding affinities are widely known by those skilled in the art. The diversity of plant sources for lectins and their sugar binding affinities is exemplified in Table 1 below (adapted from Table 3 of Van Damme et al., (1998)).

TABLE 1
Type 2 Ribosome-Inactivating Proteins and Related Lectins:
Occurrence, Molecular Structure, and Specificity

| | | | | Sequence |
|---|---|---|---|---|
| Species | Tissue | Structure^{a} | Specificity | available^{b} |
| Merolectins | | | | |
| Sambucus nigra | Bark | [P22] | NANA | Nu |
| | Fruit | [P22] | NANA | Nu |
| Hololectins | | | | |
| Sambucus nigra | Bark | II [P30]₂ | GalNAc > Gal | Nu |
| | Seed | III [P30]₂ | GalNAc > Gal | |
| | Fruit | IVI [P32]₂ | Gal/GalNAc | Nu (SNA-IV) |
| | Leaf | IVI [P32]₂ | Gal/GalNAc | Nu |
| | Leaf | IV4I [P32]₄ | Gal/GalNAc | |
| Chimerolectins | | | | |
| Abrus precatorius | Seed | [P(34 + 32)] | Gal > GalNAc | Pr, Nu (Abrin) |
| | Seed | [P(33 + 29)]₂ | Gal | Pr (APA) |
| Adenia digitata | Root | [P(28 + 38)] | Gal > GalNAc | |
| Adenia volkensii | Root | [P(29 + 36)] | Gal | |
| Cinnamonum camphora | Seed | [P(30 + 33)]_{?} | Unknown | |
| Eranthis hyemalis | Tuber | [P(30 + 32)] | GalNAc | |
| Iris hybrid | Bulb | [P(27 + 34)] | GalNAc | |
| Momordica charantia | Seed | [P(28 + 30)]₂ | Gal > GalNAc | |
| Phoradendron californicum | Plant | [P(31 + 38)] | Gal | |
| Ricinus communis | Seed | [P(32 + 34)] | Gal > GalNAc | Pr, Nu (Ricin) |
| | Seed | [P(32 + 36)]₂ | Gal >> GalNAc | Pr, Nu (RCA) |
| Sambucus canadensis | Bark | I [P(32 + 35)]₄ | NANA | |
| Sambucus ebulus | Bark | I P(32 + 37)]₄ | NANA | |
| | Leaf | [P(26 + 30)]₂ | GalNAc | |
| Sambucus nigra | Seed | Vs [P(28 + 32)]₂ | GalNAc > Gal | |
| | Bark | I [P(32 + 35)]₄ | NANA | Nu (SNA-I) |
| | Bark | I′ [P(32 + 35)]₂ | NANA | Nu (SNA-I′) |
| | Bark | V [P(26 + 32)]₂ | GalNAc > Gal | Nu (SNA-V) |
| | Fruit | If [P(32 + 36)₂ | NANA | Nu |
| | Fruit | Vf [P(26 + 32)]₂ | GalNAc > Gal | Nu |
| Sambucus racemosa | Bark | I (P(30 + 36)]₅ | NANA | |
| Sambucus sieboldiana | Bark | I (P(31 + 37)]₅ | NANA | Nu (SSA-I) |
| | Bark | [P(27 + 32)] | GalNAc > Gal | Nu (Sieboldin) |
| Viscum album | Plant | I [P(29 + 34)]₁₋₂ | Gal | |
| | Plant | II [P(29 + 34)] | Gal/GalNAc | |
| | Plant | III P(25 + 30)] | GalNAc > Gal | |
| Type 2 RIP with Inactive B chain | | | | |
| Sambucus nigra | Bark | [P(32 + 32)] | — | Nu (LRPSN) |

^{a}[PX] stands for protomer with a molecular mass of X kDa. [P(Y + Z)] indicates that the protomer is cleaved in two polypeptides of Y and Z kDa.
^{b}Pr, protein sequence; Nu, nucleotide sequence. The abbreviation in brackets refers to the sequence name used in the dendrogram (FIG. 20)].

As a further example of plant lectins contemplated herein, Table 2 below exemplifies the large number of different lectins identified from the Sambucus species alone. This group includes nigrin B, the source on NBB.

TABLE 2
Ribosome-inactivating proteins (RIPs) and lectins from Sambucus
species. Adapted from Table 1 of Ferreras et al. (2011)

| Proteins | Species | Tissues |
|---|---|---|
| Type I RIPs | | |
| Ebulitins α, β and γ | S. ebulus | Leaves |
| Nigritins f1 and f2 | S. nigra | Fruits |
| Heterodimeric type 2 RIPs | | |
| Ebulin l | S. ebulus | Leaves |
| Ebulin f | S. ebulus | Fruits |
| Ebulins r1 and r2 | S. ebulus | Rhizome |
| Nigrin b, basic nigrin b, SNA I′, SNLRPs | S. nigra | Bark |
| Nigrins l1 and l2 | S. nigra | Leaves |
| Nigrin f | S. nigra | Fruits |
| Nigrin s | S. nigra | Seeds |
| Sieboldin b | S. sieboldiana | Bark |
| Basic racemosin b | S. racemosa | Bark |
| Tetrameric type 2 RIPs | | |
| SEA | S. ebulus | Rhizome |
| SNA I | S. nigra | Bark |
| SNAIf | S. nigra | Fruits |
| SNAflu-I | S. nigra | Flowers |
| SSA | S. sieboldiana | Bark |
| SRA | S. racemosa | Bark |
| Monomeric lectins | | |
| SELlm | S. ebulus | Leaves |
| SEA II | S. ebulus | Rhizome |
| SNA II | S. nigra | Bark |
| SNAlm and SNAIVl | S. nigra | Leaves |
| SNA IV | S. nigra | Fruits |
| SNA III | S. nigra | Seeds |
| SSA-b-3 and SSA-b-4 | S. sieboldiana | Bark |
| SRAbm | S. racemosa | Bark |
| Homodimeric lectins | | |
| SELld | S. ebulus | Leaves |
| SELfd | S. ebulus | Fruits |
| SNAld | S. nigra | Leaves |

Any disease or disorder that can be treated or prevented using a therapeutic compound or agent is contemplated within the scope of the present invention. In one embodiment, the disease or disorder is one of the brain or CNS. Lysosomal diseases and (parenthetically) related enzymes and proteins associated with diseases that are contemplated within the scope of the invention include, but are not limited to, Activator Deficiency/GM2 Gangliosidosis (beta-hexosaminidase), Alpha-mannosidosis (alpha-D-mannosidase), Aspartylglucosaminuria (aspartylglucosaminidase), Cholesteryl ester storage disease (lysosomal acid lipase), Chronic Hexosaminidase A Deficiency (hexosaminidase A), Cystinosis (cystinosin), Danon disease (LAMP2), Fabry disease (alpha-galactosidase A), Farber disease (ceramidase), Fucosidosis (alpha-L-fucosidase), Galactosialidosis (cathepsin A), Gaucher Disease (Type I, Type II, Type III) (beta-glucocerebrosidase), GM1 gangliosidosis (Infantile, Late infantile/Juvenile, Adult/Chronic) (beta-galactosidase), I-Cell disease/Mucolipidosis II (GlcNAc-phosphotransferase), Infantile Free Sialic Acid Storage Disease/ISSD (sialin), Juvenile Hexosaminidase A Deficiency ((hexosaminidase A), Krabbe disease (Infantile Onset, Late Onset) (galactocerebrosidase), Metachromatic Leukodystrophy (arylsulfatase A), Mucopolysaccharidoses disorders [Pseudo-Hurler polydystrophy/Mucolipidosis IIIA (N-acetylglucosamine-1-phosphotransferase), MPSI Hurler Syndrome (alpha-L iduronidase), MPSI Scheie Syndrome (alpha-L iduronidase), MPS I Hurler-Scheie Syndrome (alpha-L iduronidase), MPS II Hunter syndrome (iduronate-2-sulfatase), Sanfilippo syndrome Type A/MPS III A (heparan N-sulfatase), Sanfilippo syndrome Type B/MPS III B (N-acetyl-alpha-D-glucosaminidase), Sanfilippo syndrome Type C/MPS III C (acetyl-CoA, alpha-glucosaminide acetyltransferase, Sanfilippo syndrome Type D/MPS III D (N-acetylglucosamine-G-sulfate-sulfatase), Morquio Type A/MPS IVA (N-acetylgalatosamine-6-sulfate-sulfatase), Morquio Type B/MPS IVB (β-galactosidase-1), MPS IX Hyaluronidase Deficiency (hyaluronidase), MPS VI Maroteaux-Lamy (arylsulfatase B), MPS VII Sly Syndrome (beta-glucuronidase), Mucolipidosis I/Sialidosis (alpha-N-acetyl neuraminidase), Mucolipidosis IIIC (N-acetylglucosamine-1-phosphotransferase), Mucolipidosis type IV (mucolipinl)], Multiple sulfatase deficiency (multiple sulfatase enzymes), Niemann-Pick Disease (Type A, Type B, Type C) (sphingomyelinase), Neuronal Ceroid Lipofuscinoses [(CLN6 disease—Atypical Late Infantile, Late Onset variant, Early Juvenile (ceroid-lipofuscinosis neuronal protein 6); Batten-Spielmeyer-Vogt/Juvenile NCL/CLN3 disease (battenin); Finnish Variant Late Infantile CLN5 (ceroid-lipofuscinosis neuronal protein 5); Jansky-Bielschowsky disease/Late infantile CLN2/TPP1 Disease (tripeptidyl peptidase 1); Kufs/Adult-onset NCL/CLN4 disease; Northern Epilepsy/variant late infantile CLN8 (ceroid-lipofuscinosis neuronal protein 8); Santavuori-Haltia/Infantile CLN1/PPT disease (palmitoyl-protein thioesterase 1); Beta-mannosidosis (beta-mannosidase)], Tangier disease (ATP-binding cassette transporter ABCA1), Pompe disease/Glycogen storage disease type II (acid maltase), Pycnodysostosis (cathepsin K), Sandhoff disease/Adult Onset/GM2 Gangliosidosis (beta-hexosaminidases A and B), Sandhoff disease/GM2 gangliosidosis—Infantile, Sandhoff disease/GM2 gangliosidosis—Juvenile (beta-hexosaminidases A and B), Schindler disease (alpha-N-acetylgalactosaminidas), Salla disease/Sialic Acid Storage Disease (sialin), Tay-Sachs/GM2 gangliosidosis (beta-hexosaminidase), and Wolman disease (lysosomal acid lipase), Sphingolipidosis, Hurmansky-Pudiak Syndrome (HPS1, HPS3, HPS4, HPS5, HPS6 and HPS7) Type 2—AP-3 complex subunit beta-1, Type 7—dysbindin), Chediak-Higashi Syndrome (lysosomal trafficking regulator protein), and Griscelli disease (Type 1: myosin-Va, Type 2: ras-related protein Rab-27A, Type 3: melanophilin).

Additional diseases (including related proteins) that may be therapeutically addressed by this invention include the neurodegenerative diseases which include but are not limited to Parkinson's, Alzheimer's, Huntington's, and Amyotrophic Lateral Sclerosis ALS (superoxide dismutase), Hereditary emphysema (al-Antitrypsin), Oculocutaneus albinism (tyrosinase), Congenital sucrase-isomaltase deficiency (Sucrase-isomaltase), and Choroideremia (Repl) Lowe's Oculoceribro-renal syndrome (PIP2-5-phosphatase). Many other genetic diseases are caused by deficiencies in specific proteins or enzymes leading to disease specific tissue and organ pathologies. ERT's or other protein replacement therapeutics may be of value for these diseases. Lectin-based carriers may facilitate protein delivery to critical organs, cells and subcellular organelles or compartments for these diseases as well.

An ERT delivery strategy has been developed that is based on the plant RTB lectin which mediates enzyme uptake and lysosomal trafficking by M6P-independent routes. This carrier supports enhanced biodistribution profiles including the treatment of currently “hard-to-treat” tissues and organs such as brain. The enzyme-RTB fusions can be produced using a plant-based bioproduction platform and thus do not contain M6P-modified glycans. The inventors have discovered that their enzyme-RTB fusions provide effective treatment even in conditions of immune-sensitized high-titer antiserum with significant implications for patients in which treatment is undermined by high titer ADA.

The present invention contemplates products in which the lectin-based carrier is operatively associated with a therapeutic component, immunogen or antigen by one of many methods known in the art. For example, genetic fusions between a plant lectin protein and a therapeutic protein can orient the lectin partner on either the C- or N-terminus of the therapeutic component, immunogen or antigen. The coding regions can be linked precisely such that the last C-terminal residue of one protein is adjacent to the first N-terminal residue of the mature (i.e., without signal peptide sequences) second protein. Alternatively, additional amino acid residues can be inserted between the two proteins as a consequence of restriction enzyme sites used to facilitate cloning at the DNA level. Additionally, the fusions can be constructed to have amino acid linkers between the proteins to alter the physical spacing. These linkers can be short or long, flexible (e.g., the commonly used (Gly₄Ser)₃ ‘flexi’ linker) or rigid (e.g., containing spaced prolines), provide a cleavage domain (e.g., see Chen et al. (2010)), or provide cysteines to support disulfide bond formation. The plant lectins are glycoproteins and in nature are directed through the plant endomembrane system during protein synthesis and post-translational processing. For this reason, production of recombinant fusion proteins comprising a plant lectin and a therapeutic protein partner may require that a signal peptide be present on the N-terminus of the fusion product (either on the lectin or on the therapeutic protein depending on the orientation of the fusion construct) in order to direct the protein into the endoplasmic reticulum during synthesis. This signal peptide can be of plant or animal origin and is typically cleaved from the mature plant lectin or fusion protein product during synthesis and processing in the plant or other eukaryotic cell. In one embodiment, a modified patatin signal sequence (PoSP) is utilized: MASSATTKSFLILFFMILATTSSTCAVD (SEQ ID NO:1) (see GenBank accession number CAA27588.1, version GI:21514 by Bevan et al. and referenced at “The structure and transcription start site of a major potato tuber protein gene” Nucleic Acid Res. 14 (11), 4625-4638 (1986)).

As used herein, compounds of the invention refers to the operatively linked agent, immunogen, or antigen with the lectin-based carrier. Compounds of the subject invention can also be prepared by producing the plant lectin and the therapeutic agent, immunogen, or antigen separately and operatively linking them by a variety of chemical methods. Examples of such in vitro operative associations include conjugation, covalent binding, protein-protein interactions or the like (see, e.g., Lungwitz et al. (2005); Lovrinovic and Niemeyer (2005)). For example, N-hydroxysuccinimde (NHS)-derivatized small molecules and proteins can be attached to recombinant plant lectins by covalent interactions with primary amines (N-terminus and lysine residues). This chemistry can also be used with NHS-biotin to attach biotin molecules to the plant lectin supporting subsequent association with streptavidin (which binds strongly to biotin) and which itself can be modified to carry additional payload(s). In another example, hydrazine-derivatized small molecules or proteins can be covalently bound to oxidized glycans present on the N-linked glycans of the plant lectin. Proteins can also be operatively linked by bonding through intermolecular disulfide bond formation between a cysteine residue on the plant lectins and a cysteine residue on the selected therapeutic protein. It should be noted that the plant AB toxins typically have a single disulfide bond that forms between the A and B subunits. Recombinant production of plant B subunit lectins such as RTB and NBB yield a product with an ‘unpaired’ cysteine residue that is available for disulfide bonding with a “payload” protein. Alternatively, this cysteine (e.g., Cys₄ in RTB) can be eliminated in the recombinant plant lectin product by replacement with a different amino acid or elimination of the first 4-6 amino acids of the N-terminus to eliminate the potential for disulfide bonding with itself or other proteins.

NBB: See GenBank accession number P33183.2, version GI:17433713 (containing subunits A and B) by Van Damme et al. and referenced at “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996). PDB ID: 3CA3 (for B subunit) by Maveyraud et al. and referenced at “Structural basis for sugar recognition, including the tn carcinoma antigen, by the lectin sna-ii from sambucus nigra” Proteins 75 p. 89 (2009).

RTB: See GenBank accession number pbd/2AAI/B, version GI:494727 (containing subunits A and B) by Montfort et al. and referenced at “The three-dimensional structure of ricin at 2.8 A” J. Biol Chem. 262 (11), 5398-5403 (1987).

In vivo administration of the subject compounds (i.e., an agent operatively linked to an LBC) and compositions containing them, can be accomplished by any suitable method and technique presently or prospectively known to those skilled in the art. The subject compounds can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intrasternal administration, such as by injection. Administration of the subject compounds of the invention can be a single administration, or at continuous or distinct intervals as can be readily determined by a person skilled in the art.

The compounds of the subject invention, and compositions comprising them, can also be administered utilizing liposome and nano-technology, slow release capsules, implantable pumps, and biodegradable containers, and orally or intestinally administered intact plant cells expressing the therapeutic product. These delivery methods can, advantageously, provide a uniform dosage over an extended period of time.

Compounds of the subject invention (i.e., an agent operatively linked to an LBC) can be formulated according to known methods for preparing physiologically acceptable compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the compound is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically-acceptable carriers and diluents which are known to those skilled in the art. Examples of carriers or diluents for use with the subject compounds include ethanol, dimethyl sulfoxide, glycerol, alumina, starch, saline, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject compounds based on the weight of the total composition including carrier or diluent.

Compounds of the invention, and compositions thereof, may be locally administered at one or more anatomical sites, optionally in combination with a pharmaceutically acceptable carrier such as an inert diluent. Compounds of the invention, and compositions thereof, may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Compounds and compositions of the invention, including pharmaceutically acceptable salts or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound of the invention in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The present invention also concerns pharmaceutical compositions comprising a compound of the invention in combination with a pharmaceutically acceptable carrier. Pharmaceutical compositions adapted for oral, topical or parenteral administration, comprising an amount of a compound constitute a preferred embodiment of the invention. The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

To provide for the administration of such dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

The subject invention also concerns kits comprising a compound and/or composition of the invention in one or more containers. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions or packaging materials that describe how to administer a compound or composition of the kit. Containers of the kit can be of any suitable material, e.g., glass, plastic, metal, etc., and of any suitable size, shape, or configuration. In one embodiment, a compound of the invention is provided in the kit as a solid, such as a tablet, pill, or powder form. In another embodiment, a compound of the invention is provided in the kit as a liquid or solution. In one embodiment, the kit comprises an ampoule or syringe containing a compound of the invention in liquid or solution form.

Mammalian species which benefit from the disclosed methods include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Other species that may benefit from the disclosed methods include fish, amphibians, avians, and reptiles. As used herein, the terms “patient” and “subject” are used interchangeably and are intended to include such human and non-human species. Likewise, in vitro methods of the present invention can be carried out on cultured cells or tissues of such human and non-human species.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

Techniques for transforming plant cells with a polynucleotide or gene are known in the art and include, for example, Agrobacterium infection, viral vectors, transient uptake and gene expression in plant seedlings, biolistic methods, electroporation, calcium chloride treatment, PEG-mediated transformation, etc. U.S. Pat. No. 5,661,017 teaches methods and materials for transforming an algal cell with a heterologous polynucleotide. Transformed cells can be selected, redifferentiated, and grown into plants that contain and express a polynucleotide of the invention using standard methods known in the art. The seeds and other plant tissue and progeny of any transformed or transgenic plant cells or plants of the invention are also included within the scope of the present invention. Likewise, techniques for expressing recombinant proteins in other eukaryotic production systems that include but are not limited to yeast, insect cell/baculovirus systems, mammalian cells, or transgenic animals is well known in the art.

Because of the degeneracy of the genetic code, a variety of different polynucleotide sequences can encode polypeptides and enzymes of the present invention. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides and enzymes of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention. Allelic variants of the nucleotide sequences encoding a wild type polypeptide of the invention are also encompassed within the scope of the invention.

Substitution of amino acids other than those specifically exemplified or naturally present in a wild type polypeptide or enzyme of the invention are also contemplated within the scope of the present invention. For example, non-natural amino acids can be substituted for the amino acids of a polypeptide, so long as the polypeptide having the substituted amino acids retains substantially the same biological or functional activity (e.g., enzymatic, or binding capability of a lectin) as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ϵ-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence of a wild type polypeptide or enzyme of the present invention are also encompassed within the scope of the invention.

Amino acids can be generally categorized in the following classes: non-polar, uncharged polar, basic, and acidic. Conservative substitutions whereby a polypeptide or enzyme of the present invention having an amino acid of one class is replaced with another amino acid of the same class fall within the scope of the subject invention so long as the polypeptide having the substitution still retains substantially the same biological or functional activity (e.g., enzymatic, or binding capability of a lectin) as the polypeptide that does not have the substitution. Polynucleotides encoding a polypeptide or enzyme having one or more amino acid substitutions in the sequence are contemplated within the scope of the present invention. Table 3 provides a listing of examples of amino acids belonging to each class.

TABLE 3

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode functional or biologically active polypeptides of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Fragments and variants of a polypeptide or enzyme of the present invention can be generated as described herein and tested for the presence of biological (e.g., binding capability) or enzymatic function using standard techniques known in the art. Thus, an ordinarily skilled artisan can readily prepare and test fragments and variants of a polypeptide or enzyme of the invention and determine whether the fragment or variant retains functional or biological activity (e.g., enzymatic activity) relative to full-length or a non-variant polypeptide.

Polynucleotides and polypeptides contemplated within the scope of the subject invention can also be defined in terms of more particular identity and/or similarity ranges with those sequences of the invention specifically exemplified herein or known in the art. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

Single letter amino acid abbreviations are defined in Table 4.

TABLE 4

| Letter Symbol | Amino Acid |
|---|---|
| A | Alanine |
| B | Asparagine or aspartic acid |
| C | Cysteine |
| D | Aspartic Acid |
| E | Glutamic Acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |
| Z | Glutamine or glutamic acid |

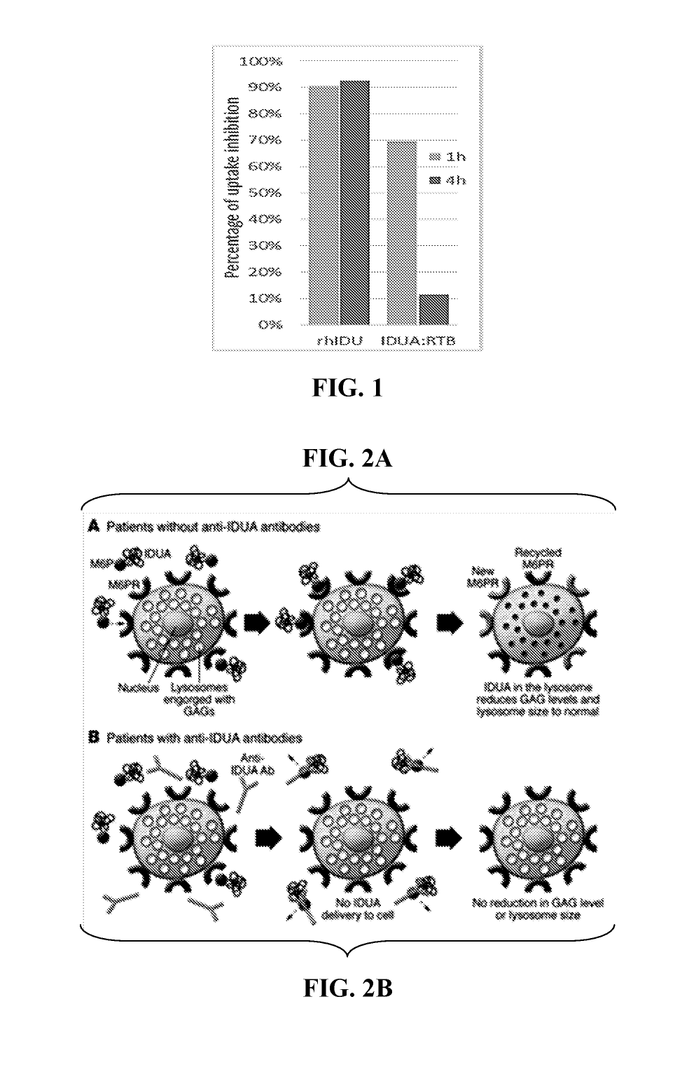

Hurler (the most severe form of MPS I) has been used extensively to delineate ERT immune sensitization and tolerance mechanisms with well-characterized mouse, rat and canine disease models. Recombinant protein comprising human α-L-iduronidase fused to the RTB lectin (IDUA:RTB) was produced and tested for uptake into cultured human MPS I patient fibroblast cells in the presence of canine serum (gift of Dr. Patricia Dickson, UCLA) containing high-titer neutralizing antibody to mammalian cell-derived iduronidase (rhIDU). Our IDUA:RTB showed greater than 90% efficiency in enzyme delivery in the presence of inhibitory canine serum, in contrast to <10% uptake of mammalian cell-derived rhIDU containing M6P FIG. 1 (discussed further below).

Animal disease models are similarly used to show that the RTB lectin fusion drugs mitigate immune-sensitization issues in vivo.

Lysosomal Storage Diseases as a Model for ADA Treatment Limitations.

Lysosomal Storage Disorders (LSD) are a group of rare genetic diseases in which a defect in a lysosomal hydrolase or other protein affects lysosomal function, resulting in accumulation of specific storage products in cell and tissues. LSDs have an estimated combined incidence of 1 in 7,000-8,000 live births. Enzyme replacement therapies (ERTs) are the treatment of choice and currently six LSDs have approved ERTs. These ERTs have been very beneficial for patient care and quality of life. However, ERTs are often compromised by immune responses—the development of neutralizing antibodies to the therapeutic enzyme. Antibody response to the ERT has been reported to hinder efficacy of the treatment in five out of the six ERTs available in patients and is prevalent in animal models used for R&D of new ERTs [3,4]. Over 90% of the patients with Hurler (MPS I), MPS VI, Pompe and Fabry develop antibody to the drug (Wang et al., 2008). Infants and children are most affected, as patients with early onset forms typically have no residual enzyme (i.e., are cross reactive immunological material negative; CRIM⁻) and quickly develop neutralizing antibodies that abrogate therapeutic correction and can precipitate severe adverse clinical effects. Impacts on Pompe patients are often cited as the most dramatic example of these reactions, where children show significant improvement in motor milestones in response to ERT only to have these effects rapidly reversed upon induction of neutralizing antibodies, leading eventually to patient death (Wang et al., 2008; Nayak et al., 2014; Kishnani et al., 2010). Tolerization protocols have been tested for patients where ERT efficacy is blocked by immune responses but these treatments are very challenging and intensive for patients and pose significant risks of infection or malignancy (Wang et al., 2008). Gene therapy approaches are currently under development for numerous LSDs as an alternative to ERTs. However, therapeutic enzymes produced via gene therapy also lead to neutralizing antibody responses in CRIM⁻ patients and animals (Xu et al., 2004) underscoring the need to develop new approaches to address the challenges imposed by immune responses to therapeutic proteins. The inventors' novel lectin-based cell-targeting carrier for therapeutic enzymes, based on fusion to the plant lectin RTB, has been shown to retain full corrective efficacy in vitro in the presence of neutralizing antibodies directed against the enzyme. Studies in MPS I patients and animals have contributed greatly to the understanding of antibody response effects and mechanisms, and provide well characterized tools and animal models to study these complex interactions.

Mucopolysaccharidosis I—ERT and Immune Sensitization.

MPS I, also called Hurler, Hurler/Scheie, or Scheie Syndrome depending on disease severity, is a chronic, progressive lysosomal storage disorder. It is caused by deficiencies in α-L-iduronidase (IDUA) resulting in pathogenic accumulation of glycosaminoglycans (GAG) in lysosomes throughout the body. Failure to effectively clear GAG leads to clinical manifestations affecting the heart, bones and joints, organs of the viscera, eyes, respiratory system, facial features, and the CNS. In its most severe form (Hurlers Syndrome), symptoms are evident in infancy leading to early death (median age 6.8 years). Current MPS I treatment options are primarily enzyme replacement therapy and/or hematopoietic stem cell transplantation. Recombinant human IDUA (rhIDU; ALDURAZYME®) produced in mammalian cells is currently available to MPS I patients and long-term ERT treatment has proven effective in reducing many of the visceral manifestations of the disease although the CNS, corneal clouding, and bone defects prevalent in MPS I are not improved. Cell uptake and lysosomal delivery of ALDURAZYME® is based on the interaction of IDUA with mannose-6-phosphate receptors (M6PR) on target cells.

Among MPS I patients receiving ALDURAZYME®, 91% develop anti-rhIDU IgG antibodies. Evidence of reduced drug efficacy includes elevated urinary GAG excretion in patients with high antibody titers to rhIDU (Wraith et al., 2007). In animal models, development of anti-rhIDU antibodies alters the biodistribution in organs and organelles (Turner et al., 2000) and reduces delivery of enzyme to organs by interfering with the M6P receptor-mediated uptake mechanism (Dickson et al., 2008; Glaros et al., 2002; Ponder, 2008) (FIGS. 2A and 2B).

Lectin-Based Carriers Provide New Mechanisms of ERT Uptake and Lysosomal Trafficking.

We have developed the RTB plant lectin as a novel carrier for lysosomal enzymes. RTB is the non-toxic carbo-hydrate-binding B subunit of the plant type II AB toxin, ricin. RTB facilitates uptake by targeting cell surface glycoproteins and glycolipids with β-1,4-linked galactose or galactosamine residues. These are abundant on mammalian cells (Sandvig et al., 2014; Olsnes, 2004), providing access to a broad array of cells.

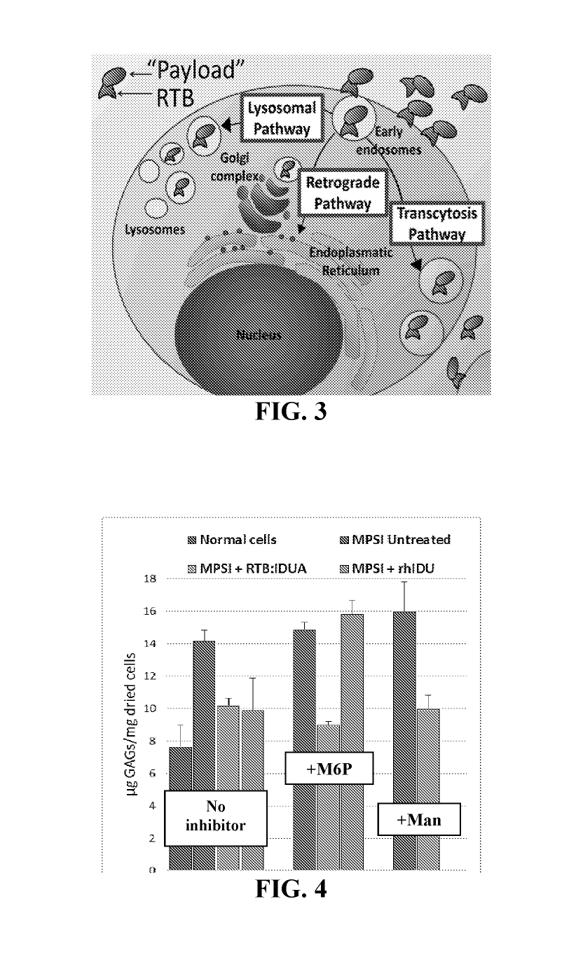

RTB enters cells by at least 6 different endocytotic routes including both absorptive- and receptor-mediated mechanisms (Sandvig et al., 2011; Sandvig et al., 1999; Simmons et al., 1986; Frankel et al., 1997). Upon endocytosis, RTB traverses preferentially to lysosomes (FIG. 3) or cycles back to the cell membrane (transcytosis pathway), with less than 5% moving “retrograde” to the endoplasmic reticulum (route for RTA toxin delivery) (Olsnes, 2004; Van Deurs et al., 1986). RTB fusions (both RTB:IDUA and IDUA:RTB) were produced using a transient plant-based expression system.

Lectin-IDUA Fusions Corrects GAG Levels by M6P Receptor-Independent Mechanisms.

Plant-made RTB:IDUA and IDUA:RTB fusion proteins retain both RTB lectin binding activity and IDUA enzyme activity. Unlike mammalian cells, plant cells do not possess the enzymatic machinery to make M6P-modified glycans (He et al., 2013). Thus, mammalian cell uptake of these fusion products is solely mediated by the RTB lectin. To demonstrate this, purified RTB:IDUA product was used to treat MPS I/Hurler patient fibroblasts. Treatment with the RTB fusion product resulted in GAG reduction to “normal” levels comparable to control mammalian cell-derived rhIDU. As shown in FIG. 4, RTB-mediated delivery of IDUA was independent of mannose-6-phosphate receptors (in contrast to rhIDU) and high-mannose receptors (Acosta et al., 2015). Our in vitro data indicate that RTB efficiently delivers functional enzyme into cells and mediates disease substrate clearance by mechanisms that are fundamentally different than all current ERTs for LSDs. In vivo data in MPS I mice indicate biodistribution of IDUA:RTB to multiple visceral organs following a single tail-vein injection and disease correction based on GAG levels, loss of splenomegaly, and improved memory/learning following multiple weekly injections (Acosta et al., 2016; Ou et al., 2016). Thus, IDUA:RTB functions as a highly effective ERT.

Plant-Based Bioproduction.

Our IDUA:RTB fusion is produced in a rapid plant-based transient expression system (Whaley et al., 2011; Komarova et al., 2010). Plants provide advantages in safety (no adventitious viral contamination issues) and cost of manufacture and have been shown to synthesize fully functional human lysosomal enzymes (U.S. Pat. No. 5,929,304). In 2012, FDA-approved plant-derived Elelyso (Protalix/Pfizer) for treatment of Gaucher patients. Elelyso is being offered at 75% of the cost of the CHO-derived product. This product has been administered to patients for 8 years (Aviezer et al., 2009) and shows no increase in immunogenicity compared to mammalian cell-derived glucocerebrosidase (Grabowski et al., 2014) and is well tolerated by patients switching from animal-cell-derived products (Grabowski et al., 2014; Pastores et al., 2013).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention.

These examples should not be construed as limiting. All percentages are by weight and all solvent mixture proportions are by volume unless otherwise noted.

### Example 1

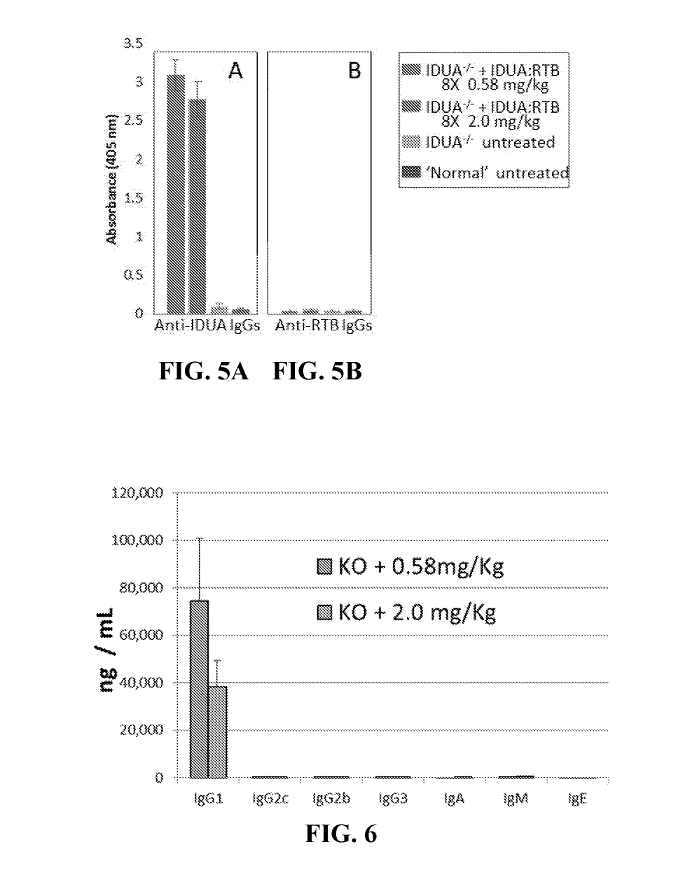

Patients develop immune responses to protein-based treatments that can lead to reduced efficacy (or abolishment of efficacy), altered biodistribution, and, in some cases, life-threatening adverse immune responses. BioStrategies has been developing the lectin component (non-toxic carbohydrate-binging B-subunit) of plant AB toxins as carriers for associated human proteins. These include human lysosomal enzymes capable of treating lysosomal diseases by providing the proteins that are genetically deficient termed “enzyme replacement therapies” (ERTs). Our lead plant lectin is RTB (B-subunit of ricin). RTB binds to galactose and galactosamine residues that are abundant on the surface of human cells, triggers endocytosis, and directs trafficking of associated proteins using the endosome to lysosome and transcytosis pathways. We have produced a variety of RTB fusion proteins linking RTB to enzymes and other proteins using a plant-based protein bioproduction platform. We have demonstrated that RTB effectively delivers the associated “payload” proteins into mammalian cells both in vitro and in multiple in vivo mouse disease models. Once bound to the cell surface, RTB triggers adsorptive-mediated endocytosis and transcytosis and has been shown to support broad in vivo biodistribution including cells of so-called “hard-to-treat” tissues and organs (e.g., brain, heart, lung). Analyses in mice and in human cells have revealed two unexpected features of the plant lectin carrier that suggests highly unique and useful interactions with the immune system. 1) Although serum antibodies were developed against the “payload” protein (two examples—human iduronidase as IDUA:RTB fusion and the green fluorescent protein as RTB:GFP) following multiple administrations in mice, no antibodies directed against the RTB lectin were detected (see FIGS. 5A and 5B). And 2) if the IDUA:RTB protein was exposed to neutralizing antibodies to the IDUA component (from animals that had lost the ability to respond to corrective enzyme due to the high-titer of neutralizing anti-IDUA antibodies in their serum) and then used to treat disease cells, the RTB carrier was still able to support cell uptake and lysosomal delivery, and provide active enzyme that degraded the disease substrate to correct the lysosomal disease cellular phenotype.

Thus plant lectins provide novel and unanticipated advantages as “stealth carriers” for associated therapeutic macromolecules that may elicit anti-drug immunogenicity. These advantages include:

Enzymes fused with RTB will restore efficacy in patients that have already developed neutralizing anti-ERT or anti-drug immune responses that undermine treatment efficacy; and

Patients initiated with treatment comprising RTB fused to a therapeutic component will retain treatment efficacy even if serum antibodies are developed to the therapeutic macromolecule; and

Patients can be treated with additional therapeutic agents that employ the RTB carrier providing similar distribution and trafficking patterns that would not be disrupted by potential anti-carrier antibodies.

Potential Product Immunogenicity.

Development of anti-drug immune responses has been challenging for many LSD patients receiving currently approved ERT biologics. BioStrategies has initiated several studies to assess immune impacts of our RTB:enzyme fusions. Initial results indicate that RTB not only lacks significant immunogenicity itself, but also may provide advantages as an enzyme carrier in patients with inhibitory antibodies to their current ERT drug.

We demonstrate here in FIG. 1 that RTB delivers corrective ERT doses into cells even in the presence of inhibitory levels of anti-ERT neutralizing antibodies. In vivo results (FIGS. 5A and 5B) on induction of serum antibodies in MPS I (IDUA^{-/-}) mice following 8 weekly injections of plant-made IDUA:RTB (Acosta et al., 2016; Ou et al., 2016) have also been performed. These data indicate that MPS I mice develop anti-IDUA antibody titers analogous to that seen following treatment with mammalian cell derived IDUA (Ou et al., 2014; Baldo et al., 2013). In contrast, mice did not develop antibodies directed against RTB (see FIG. 5B) or against the glycans of plant-made IDUA.

Humoral response was further analyzed by isotyping the antibodies present in the terminal serum. More than 99% of the immunoglobulins produced belongs to the IgG1 subclass, a typical response against protein and peptide antigens. Insignificant production of other isotype subclasses suggests that the response is not triggered by the carbohydrates/polysaccharides present in the protein (IgG2, IgA) or due to an allergic reaction against the therapeutic protein (IgE) FIG. 6.

### Example 2

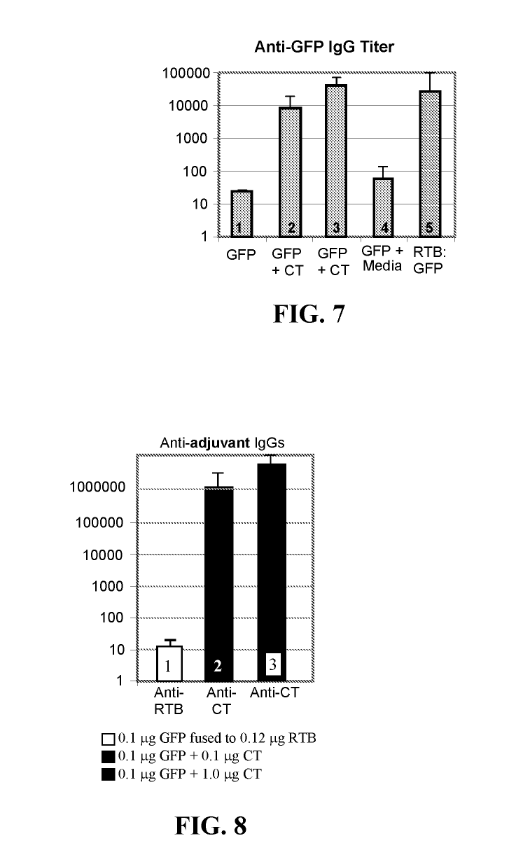

Mucosal vaccines, those delivered intranasally or orally, can be highly effective in triggering both systemic and mucosal immune responses. However, of the 22 vaccines currently in routine use for non-biowarfare infectious diseases, 20 are delivered by injection and stimulate only systemic immunity. Although potential “protective antigens” have been identified for many disease agents including Category A and B agents, they generally require an “adjuvant” or specific carrier in order to trigger a strong immune response. For injectable vaccines, alum (aluminum hydroxide; an irritant) and more recently, MF59 (a mix of squalene and surfactants) are the only adjuvants currently approved by the FDA for use in humans. However, these compounds are not effective adjuvants for mucosal vaccines. Cholera toxin has been the “gold standard” mucosal adjuvant for nasal and oral delivery of vaccines in rodents but is not approved for humans because of associated toxicity. We have developed the non-toxic carbohydrate-binding subunit of ricin toxin (B subunit; RTB) as a mucosal adjuvant and carrier and demonstrated that RTB provides vaccine adjuvancy equivalent to cholera toxin for nasal delivery (Medina-Bolivar et al., 2003). This galactose/galactosamine-binding lectin binds to human mucosal surfaces (including a high affinity for M-cells), and thus functions to deliver fused antigens directly to immune-responsive cells of the mucosa. The efficacy of RTB as an antigen delivery system for mucosal vaccines was demonstrated using the green fluorescent protein (GFP) as a model antigen. GFP was genetically fused to RTB and expressed in tobacco plants and in root cultures derived from these plants (Medina-Bolivar et al., 2003). Tobacco-synthesized RTB:GFP, a 62 kD glycoprotein which retains both GFP fluorescence and RTB carbohydrate binding specificity, was affinity-purified from the media of root cultures using a galactosamine resin and used for nasal immunization of mice. The immune responses of mice immunized intranasally with GFP alone, GFP plus cholera toxin adjuvant, or affinity-purified RTB:GFP from tobacco were compared. As shown in FIG. 7, RTB:GFP triggered significant increases in GFP-specific serum IgGs. This strong humoral response was comparable to that observed following GFP immunization with cholera toxin adjuvant. GFP at the same concentrations but without an adjuvant was non-immunogenic. Induction of higher levels of IgG₁ than IgG₂ₐ following RTB:GFP immunization suggested that RTB, like CT, mediates primarily a Th2 response. Serum and fecal anti-GFP IgAs were also elevated at levels equivalent to that seen with cholera toxin as adjuvant (Medina-Bolivar et al., 2003), supporting the effectiveness of RTB as an adjuvant and antigen carrier to the mucosa.

RTB functions as a “stealth” adjuvant. Most protein-based mucosal adjuvants (e.g., CT, LT, CT/LT derivatives, mistletoe lectin, proteosomes) are strongly immunogenic eliciting high serum titers of anti-adjuvant antibodies. This has raised concerns of reduced adjuvancy in later boosts or as a component of a distinct vaccine.

More critically, concerns have been raised as to whether reuse of these adjuvants in a subsequent vaccine might trigger adverse hypersensitive immune responses. In preliminary studies, RTB shows striking differences in its intrinsic immunogenicity compared to CT (and reports of LT and mistletoe lectin adjuvants). As shown in FIG. 8, antibodies specific to RTB were not detected in serum of mice immunized intranasally with RTB:GFP even though high titers of anti-GFP antibodies were induced (see FIG. 7). In contrast, high levels of anti-CT antibodies were present in mice immunized with CT+GFP (FIG. 8). This is not due solely to the low level of RTB used (100 ng/dose) since CT at 100 ng/dose was highly immunogenic. Thus, RTB may be unusually non-immunogenic as an adjuvant. In support of this, several groups have tried to use RTB as a protective antigen to develop vaccines against ricin toxin (RTA+RTB) without success (N. Mantis, Children's Hosp. Boston; G. Glenn, Walter Reed Army Inst. of Research, pers. comm.). To further delineate this intriguing feature of RTB and expand its utility as an adjuvant and mucosal carrier, experiments are designed to assess the levels of anti-RTB IgGs following mucosal immunizations with higher doses of RTB:GFP fusion, additional boosts extended over longer periods, and following administration of a second antigen-RTB fusion. This “stealth” characteristic of RTB has significant implications with respect to regulatory acceptance and “reuse” potential in multiple vaccines. For example, RTB could serve as carrier for a subunit Ebola antigen vaccine with protective immunity elicited by multiple vaccination/boost protocols to gain robust protection against the Ebola virus. The same patient could be immunized later with a vaccine for protection against Zika virus using a Zika antigen associated with the RTB carrier. Since RTB itself is non-immunogenic, there would be no immune suppression caused by the previous exposure to RTB that could undermine the desired immune response to the Zika antigen.

### Example 3

RTB delivers IDUA in presence of inhibitory anti-rhIDU antibodies. Neutralizing canine serum from rhIDU-immunized animals inhibits ERT uptake in human MPS I fibroblast by interfering with the M6P receptors (Dickson et al., 2008). To determine if RTB will deliver corrective doses of human IDUA into disease cells in the presence of neutralizing antibodies, we compared cell uptake of IDUA:RTB versus rhIDU following pre-incubation with neutralizing canine serum (provided by P. Dickson). Consistent with previous reports (Dickson et al., 2008), serum from high anti-rhIDU titer dogs inhibited uptake of mammalian cell-derived rhIDU by greater than 90% (FIG. 1). In contrast, IDUA:RTB showed 70% inhibition at 1 hr but by 4 hr, 90% of the IDUA:RTB was successfully taken up into cells (FIG. 5). These results suggest that RTB delivery technology will facilitate delivering of corrective enzyme in patients that have developed uptake-blocking antibodies to rhIDU. This should be broadly applicable to other disease treatments where ADA impacts cellular uptake and trafficking of drug.

### Example 4

The RTB carrier module is itself non-immunogenic. In one example, wildtype mice were transnasally vaccinated and boosted 2-3 times with RTB:GFP fusions as described in (Medina-Bolivar et al., 2003). They were vaccinated in the presence of Freund's adjuvant in order to elicit strong immunity. Although all vaccinated mice developed strong antibody titers to the GFP “cargo”, essentially no antibodies above background were detected against RTB. In two other examples, knockout mice for two different lysosomal diseases were treated with lysosomal enzyme:RTB fusions for 4 to 6 weeks at various doses in trials that demonstrated drug efficacy in disease correction. Serum was collected at multiple times and tested for induction of anti-drug antibodies. Antibodies (IgGs) against the human lysosomal enzyme component were detected with levels comparable to those reported in the literature when mice are treated with the mammalian-cell-derived enzyme alone (i.e., no RTB). In contrast, anti-RTB titers in the same animals were very low (essentially background).

These data indicate that although RTB carries an immunogenic protein cargo, the carrier itself shows very low immunogenicity—is a “stealth” carrier. Because RTB is capable of directing cell uptake even when the cargo is bound by serum antibodies (see FIG. 1), RTB-Enzyme fusions retain long-term efficacy with chronic administration.

Note—RTB is one of a class of lectin that function similarly although the sugar binding specificity may differ among lectins.

MPS I is one example of many therapies that could benefit from technology

### Example 5

Lectin-mediated delivery of ERT and lysosomal disease correction occurs in animals previously immune-sensitized to the ERT drug. The potential of our lectin-based carrier to effectively deliver corrective enzyme is tested in MPS I/Hurler mice that are immune-sensitized to the rhIDU drug. 1) MPS I (idua^{-/-}) mice are immunized with rhIDU to yield animals with high-titer neutralizing anti-rhIDU antibodies). 2) Both sensitized and naïve mice are provided weekly enzyme treatments of either rhIDU or IDUA:RTB and impacts on IDUA activity and GAG levels in selective organs and fluids is compared (FIG. 9).

A. Develop MPS I Mice that are Immune-Sensitized to the rhIDU ERT Product.

Rationale. Our first goal is to establish the immunization protocol to produce idua^{-/-} mice with high-titer anti-rhIDU antibodies. Two strategies have previously been used to develop immune-sensitized idua^{-/-} animals: 1) weekly rhIDU administrations at the human dose (0.58 mg rhIDU/Kg) or higher, reflecting patient treatment protocols¹, or 2) adjuvanted immunizations with ERT, patterned after vaccination protocols (Turner et al., 2000; Clements et al., 1985; Ashton et al., 1992). The latter approach requires less enzyme, fewer administrations, and provides a greater proportion of high-titer animals (100% (Turner et al., 2000)) compared to non-adjuvanted rhIDU alone and will thus be used for these studies. We will test 2 rhIDU doses, 1 μg and 5 μg/mouse, administered with Freund's complete (prime) or Freund's incomplete (3 boosts) adjuvant. The 5 μg/mouse dose matches immunization protocols reported in rats (50 μg rhIDU dose equivalent to ˜0.2 mg/kg) which yielded 100% high-titer animals (Turner et al., 2000).

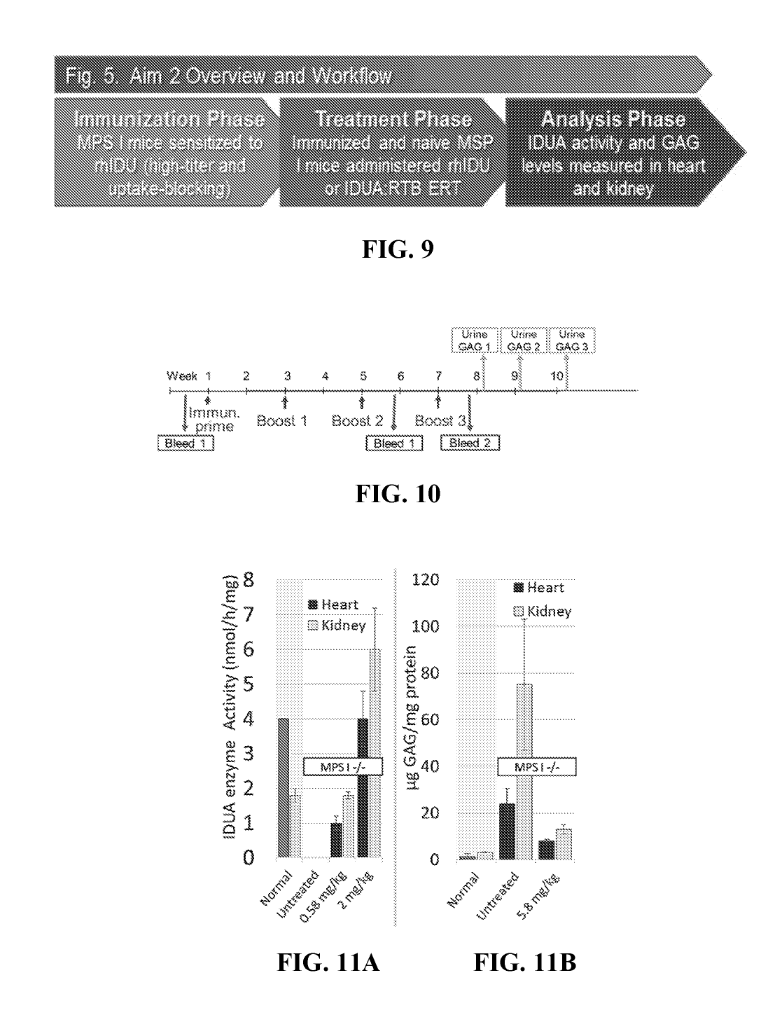

The MPS 1 mice effectively model human Hurler syndrome with similar behavioral disease development, lack of IDUA catalytic activity, considerable GAG accumulation in internal organs, and 3-fold higher urinary GAG levels than normal mice (Wang et al., 2010; Ou et al., 2014). IDUA-cross reactive protein is not detectable by westerns (Keeling, UAB, pers. comm.). Serum anti-rhIDU IgG levels is measured by ELISAs before immunization and in samples collected following the 2^{nd} and 3^{rd} boosts (see FIG. 10). The presence of neutralizing antibodies is assessed based on serum-mediated inhibition of enzyme uptake into human MPS I fibroblasts (see FIG. 1). After the final boost, urine is collected weekly for three weeks from immunized and age-matched non-immunized MPS I mice for GAG level determinations (see FIG. 10). Urinary GAG levels provide a non-invasive way to assess MPS I disease and correction (Dickson et al., 2008). Based on these initial trials on a small cohort of animals, we select a dose and boost schedule for a larger cohort to support Aim 2 studies, which compare enzyme treatments in immunized and non-immunized animals. For this trial, 18 idua^{-/-} mice (6-8 wks old) are immunized with the selected adjuvanted protocol. Mice are bled 6 days after the final boost and their sera analyzed for anti-rhIDU antibodies and fibroblast uptake neutralization. Confirmed high-titer mice are then used in conjunction with non-immunized mice for disease treatment studies.

Immunizations.

For initial immunization protocol assessment, two groups of idua^{-/-} mice (6-8 weeks; n=4) are immunized subcutaneously with either 1 μg or 5 μg rhIDU (assuming 25 μg mouse at either 0.04 or 0.2 mg/kg dose). Initial immunization is adjuvanted 1:1 with Freund's complete adjuvant with boosts at 2-week intervals using enzyme plus Freund's incomplete adjuvant (administration volumes of 100-150 μl).

Animals are bled (orbital or tail vein) prior to first immunization and 6 days after 2^{nd} and 3^{rd} boosts. A larger cohort (n=18) is subsequently immunized at a single selected rhIDU dose and boosting schedule to support Aim 2 studies. These animals are bled 6 days after final boost.

Assessment of Anti-rhIDU Serum Titers.

Serum titers are analyzed by ELISA as described (Kakkis et al., 2004). Briefly, 96-well plates are coated with rhIDU protein (200 ng/well), washed, and incubated with a serum dilution series. Bound antibodies are detected with AP-conjugated rabbit anti-mouse-IgG antibodies (absorbance 405 nm). Data are presented based on OD units/ml serum based on dilutions read within the linear range. High-titer animals are defined as those having OD units greater than 5 OD units/ml serum.

Uptake Inhibition Assays in MPS I Fibroblasts.

To determine if high-titer serum from sensitized animals contains antibodies that block M6P-mediated uptake, an antibody-mediated uptake inhibition assay is performed as described (Dickson et al., 2008) (see also FIG. 1 that tested uptake in presence of canine serum). rhIDU is pre-incubated in media with mouse serum for 1 hour prior to addition to cells. Several serum dilutions are tested (1:1000, 1:500); for canine serum, 1:1000 dilution provided >90% inhibition of rhIDU uptake¹ (FIG. 1). After incubation with confluent cultures, cells are harvested, and intracellular IDUA activity is measured in cell lysates using standard fluorometric assays with 4-MU-iduronide. Percentage of uptake inhibition is calculated by comparing intracellular IDUA activity of cells incubated with rhIDU+/−serum.

Measurement of Urinary GAG.

Urine samples are collected from individual mice over a 24 hr period (e.g., using metabolic cages), sterile filtered, and stored at 4° C. until assayed. GAG content is quantified using the dimethylmethylene blue chloride (DMMB) as described (Wang et al., 2010; De Jong et al., 1992). GAG levels are normalized to creatinine and expressed as mg GAG per mg creatinine (Wang et al., 2010; De Jong et al., 1992).

These studies establish specific immunization parameters (rhIDU immunogen dose, number of boosts) required to yield strong uptake-blocking immune response in MPS I mice. These conditions are then used to produce a cohort of rhIDU-sensitized mice for disease treatment.

B. Compare IDUA Activity and GAG Levels in Selected Tissues in rhIDU-Sensitized Mice Following Short-Term Treatments with Either Commercial rhIDU or IDUA:RTB.

Rationale.

The hypothesis that IDUA:RTB can deliver corrective doses of IDUA enzyme in animals with high-titer anti-rhIDU antibodies is tested. The overall strategy is summarized in FIG. 9. The immunization phase (described above) produces rhIDU-immunized MPS I mice with immune sensitization status qualified by serum anti-rhIDU antibody levels and cell uptake inhibition assays. In the treatment phase, confirmed high-titer immunized and age-matched non-immunized MPS I mice are treated intravenously weekly for a total of 4 treatments with rhIDU or plant-made IDUA:RTB (see Table 5). Each treatment provides the human therapeutic dose equivalent of 0.58 mg IDUA/kg. E.g., for a 25 μg mouse, this are 14.5 μg rhIDU (˜85 kDa) or 20.5 μg IDUA:RTB (˜120 kDa). IDUA enzyme activity and GAG levels are assessed in heart and kidney 4 days after final therapeutic treatment.

TABLE 5
Animal treatments.

| Animal | Number | Immunization | ERT Treatment |
|---|---|---|---|
| MPS I (idua^{-/-}) | n = 3-6* | rhIDU-immunized | rhIDU |
| MPS I (idua^{-/-}) | n = 3 | None | rhIDU |
| MPS I (idua^{-/-}) | n = 3-6* | rhIDU-immunized | IDUA:RTB |
| MPS I (idua^{-/-}) | n = 3 | None | IDUA:RTB |
| MPS I (idua^{-/-}) | n = 3-6* | rhIDU-immunized | None |
| MPS I (idua^{-/-}) | n = 2 | None | None |
| Normal (idua^{+/+}) | n = 2 | None | None |

*6 mice will be immunized; at least 3 will be treated and/or analyzed

Heart and kidney are selected as the primary organs for these analyses based on the following: In multiple studies assessing impacts of anti-drug antibodies on enzyme therapy or testing potential tolerization strategies, alterations in therapeutic enzyme biodistribution provided the most reliable short-term indicator of immune-sensitization (Dickson et al., 2008; Glaros et al., 2002). However, impacts on specific organs differ-organs such as liver that are rich in macrophages and reticuloendothelial cells may actually have elevated IDUA levels in high-titer individuals, putatively linked with antibody-directed (as opposed to M6P-directed) uptake. In contrast, kidney, heart and lung consistently show reduced IDUA activity and higher GAG levels in rhIDU-sensitized animals compared to low-titer (non-immunized or immune-tolerized) animals (Dickson et al., 2008; Glaros et al., 2002), In recent experiments, the Dickson group demonstrated that heart and kidney rhIDU activity levels were reduced by more than 50% in high-titer (3-30 OD units/ml) MPS I mice compared to low-titer (<1 OD unit/ml) mice following 4 weekly doses (Dickson, pers. comm.; manuscript submitted). Heart and kidney produced the most dramatic and statistically significant differences in high-versus low-titer animals. Therefore, initial analyses are restricted to these organs. Follow-on studies provide more thorough investigations of IDUA:RTB biodistribution and efficacy in immune-sensitized mice.

The selection of heart and kidney for the analyses is also supported by our in vivo studies with IDUA:RTB Initial trials with IDUA:RTB administered to MPS I mice demonstrate that our plant-made product is effectively taken up by heart and kidney, as well as other organs (Acosta et al., 2016; Ou et al., 2016). As shown in FIG. 11A, substantial IDUA activity was detected in these organs 24 hr after administration (levels in kidney were equivalent to WT). Because this was the first in vivo administration of IDUA:RTB, we also treated 3 mice with a 10× dose (5.8 mg IDUA equivalents/kg) and monitored them for 5 days for any adverse effects. No signs of toxicity were observed and, at endpoint, these mice were analyzed for tissue GAG levels. Significant GAG clearance was seen in kidney and heart (FIG. 11B) as well as liver and spleen (data not shown). These results provide confidence that IDUA activity and IDUA:RTB-mediated GAG reduction are detectable in heart and kidney tissue in non-immune MPS I mice and that the experimental design should be effective in demonstrating that IDUA:RTB, but not rhIDU, can mediate GAG reduction in the presence of high antibody titers.

Production of Plant-Derived IDUA:RTB.

At BioStrategies, bioproduction and purification protocols for IDUA:RTB has been established to generate defined, well characterized protein to support mouse biodistribution and efficacy studies. Transient expression in Nicotiana benthamiana (Whaley et al., 2011; Komarova et al., 2010) is accomplished by vacuum infiltration of intact plants with Agrobacterium tumefaciens carrying the IDUA:RTB construct. After four days, leaves are harvested and a simple 3-step purification protocol yields product with >95% purity. Established quality control protocols for the final product include quantification of lectin binding activity, IDUA enzyme units, protein concentration by absorbance at 280 nm, and endotoxin levels using a modified Limulus Amebocyte Lysate (LAL) assay (detects to 0.005-1 EU/ml).

Enzyme Replacement Treatments.

High-titer immunized mice (see above) and age-matched naïve mice are administered IDUA enzyme (0.58 mg IDUA equivalent/kg in PBS (<150 μl) rhIDU or IDUA:RTB by tail vein injection) weekly starting two weeks after the immunized group receives the final boost. Mice are monitored carefully for injection-related stress. After 4 treatments, mice are euthanized, perfused, and selected organs isolated, weighted and snap-frozen. Various control groups (see Table 5) are processed in parallel. Analyses of heart and kidney IDUA activity and GAG levels in tissue homogenates are described previously (Wang et al., 2010; Ou et al., 2014).

Statistical Analyses.

Evaluation of differences between samples are analyzed using Tukey test for comparisons between paired samples and two way analysis of variance (ANOVA) for comparisons between three or more samples. Statistical significant level are set at p<0.05. This study represents a preliminary proof-of-concept feasibility test. More extensive preclinical assessments will further support power statistical analyses.

These studies compare IDUA:RTB and rhIDU enzyme treatments on rhIDU-immunized and non-immunized MPS I mice and analyze impacts on IDUA activity and GAG levels in kidney and heart (e.g., FIGS. 11A and 11B). These represent technically straight-forward procedures for which the protocols and expertise are established and the strategy is well-supported by the literature. These studies show IDUA:RTB is able to circumvent high anti-rhIDU serum titers and successfully deliver corrective enzyme in high-titer mice. The definitive data are the comparison of heart/kidney IDUA activity and GAG levels in immunized mice that are treated with IDUA:RTB versus rhIDU. IDUA:RTB treated mice show 1) higher IDUA activity and significant GAG correction (e.g. 50% of untreated MPS I mice) and 2) greater GAG reduction in heart and kidney than rhIDU-treated animals. Based on previous studies, including our own preliminary data with IDUA:RTB, 4 weekly treatments at the proposed ERT dose provided sufficient differences in treated versus untreated GAG levels to distinguish ERT efficacy among the groups (Dickson et al., 2008; Ou et al., 2014). We have included various control groups (untreated MPS I mice, immunized/untreated MPS I mice, non-immunized/treated mice) to 1) document that both the rhIDU and IDUA:RTB enzymes are functional and provide the expected treatment outcomes in non-immunized mice and 2) provide baseline “normal” and baseline “MPS I disease” levels from age-matched siblings. Both therapeutic enzymes are qualified (quantity, purity, IDUA activity) before administration, but experiments are repeated with new qualified enzyme or higher ERT doses are tested if control treatments (naïve MPS I mice) fail to show the expected reduction in heart/kidney GAG levels.

These studies delineate the novel delivery mechanisms of IDUA:RTB to circumvent the inhibition of therapeutic efficacy imposed by circulating anti-rhIDU neutralizing antibodies. Additional studies assess: IDUA:RTB biodistribution and pharmacodynamics in immunized and non-immunized animals; IDUA:RTB immunogenicity; proof of concept in other LSD diseases including Pompe, GM1 gangliosidosis, Hunter and other diseases and treatments where the prevalence of immune responses is undermining efforts to bring new ERTs or gene therapy options to patients (Wang et al., 2008; Kishnani et al., 2010; Xu et al., 2004).

### Example 6

Example 5 highlights utility for the lectin carrier—therapeutic/bioactive molecule fusion to effectively treat individuals that have previously developed ADA to the therapeutic entity that undermines treatment efficacy. Example 6 provides additional advantages of the technology in treatment of “naïve” individuals such that even if they develop ADA antibodies during chronic treatment, the carrier continues to delivery to disease-critical cells and tissues and the individual avoids ADA-mediated decline in treatment efficacy.

Disease knockout mice, such as the MPS I or Pompe mouse models, are treated with a normal or 10× normal corrective doses (e.g. 0.58 mg/kg and 5.8 mg/kg IDUA equivalents for MPS I mice) at weekly intervals using either mammalian cell-derived enzyme (e.g., rhIDU) or lectin-carrier fused enzyme (e.g., IDUA:RTB). Urine GAG levels (or equivalent readouts associated with the specific disease) are assessed weekly and serum antibodies assessed every 3 weeks. The number of animals that show improvement (e.g., low urine GAG) followed by decline (e.g., elevated urine GAG) correlated with anti-enzyme antibody titers are compared among treatment groups. At the treatment stage showing a significant difference between the enzyme vs. the enzyme:RTB treatment, selected animals will be sacrificed to assess liver, kidney, spleen and heart enzyme and GAG levels. The utility of RTB as a long-term carrier is exemplified by continuous treatment efficacy that does not show the ADA-associated decline in treatment efficacy.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,938,949
U.S. Pat. No. 5,661,017
U.S. Pat. No. 5,705,484
U.S. Pat. No. 5,929,304
U.S. Pat. No. 6,884,419
U.S. Pat. No. 7,011,972
U.S. Pat. No. 7,410,779
U.S. Pat. No. 7,867,972
U.S. Pat. No. 7,964,377
Acosta W, Ayala J, Dolan M C, Cramer C L “RTB Lectin: a novel receptor-independent delivery system for lysosomal enzyme replacement therapies” Scientific Reports, 2015, 5:14144 (11 pages).
Acosta W, Ou L, Ayala J, Condori J, Katta V, Flory A, Martin R, Radin J, Whitley C, Cramer C, Radin D. 2016 Lectin-mediated delivery of α-L-iduronidase: A novel approach for MPS I enzyme replacement therapy. Mol. Genet. Metab. Vol. 117 (2) p. S14-S15
Altschul, S. F. et al. (1990) “Basic Local Alignment Search Tool” J. Mol. Biol. 215:402-410.
Altschul, S. F. et al. (1997) “Gapped BLAST and PSI-BLAST: A New Generation of Protein Database Search Programs” Nucl. Acids Res. 25:3389-3402.
Ashton L, Brooks D, McCourt P, Muller V, Clements P, Hopwood J. Immunoquantification and enzyme kinetics of alpha-L-iduronidase in cultured fibroblasts from normal controls and mucopolysaccharidosis type I patients. Am J Hum Genet. 1992; 50(4):787-794.
Aviezer D, Brill-Almon E, Shaaltiel Y, Hashmueli S, Bartfeld D, Mizrachi S, Liberman Y, Freeman A, Zimran A, Galun E. A Plant-Derived Recombinant Human Glucocerebrosidase Enzyme-A Preclinical and Phase I Investigation. PLoS One. 2009; 4(3):e4792.
Baldo G, et al. (1990) “Enzyme replacement therapy started at birth improves outcome in difficult-to-treat organs in mucopolysaccharidosis I mice” Molec. Genet. Metab. 109(1):33-40.
Bevan M, Barker R, Goldsbrough A, Jarvis M, Kavanagh T, Iturriaga G “The structure and transcription start site of a major potato tuber protein gene” Nucleic Acid Res. 1986, 14(11):4625-4638.
Chen X, Bai Y, Zaro J L, Shen W C “Design of an in vivo cleavable disulfide linker in recombinant fusion proteins” BioTechniques, 2010, 49(1):513-518.
Clements P, Brooks D, Saccone G, Hopwood J. Human α-L-iduronidase. Eur J Biochem. 1985; 152:21-28.
Condori J, Acosta W, Ayala J, Katta V, Flory A, Martin R, Radin J A, Cramer C L, Radin D N. 2016. Enzyme replacement for GM1-gangliosidosis: Uptake, lysosomal activation, and cellular disease correction using a novel β-galactosidase:RTB lectin fusion. Molec. Genet. Metab. 117:199-209.
De Jong J, Wevers R, Liebrand-van Sambeek R. Measuring urinary glycosaminoglycans in the presence of protein: An improved screening procedure for mucopolysaccharidoses based on dimethylmethylene blue. Clin Chem. 1992; 38(6):803-7.
Dickson P, Peinovich M, McEntee M, Lester T, Le S, Krieger A, Manuel H, Jabagat C,
Passage M, Kakkis E D “Immune tolerance improves the efficacy of enzyme replacement therapy in canine mucopolysaccharidosis I” J Clin Invest., 2008, 118(8):2868-2876.
Frankel A, Fu T, Burbage C, Tagge E, Harris B, Vesely J, Willingham M, Lectin-deficient ricin toxin intoxicates cells bearing the d-mannose receptor. Carbohydrate R. 1997; 300:251-258.
Glaros E, Turner C, Parkinson E, Hopwood J, Brooks D. Immune response to enzyme replacement therapy: single epitope control of antigen distribution from circulation. Mol Genet Metab. 2002; 77(1-2):127-135.
Grabowski G, Golembo M, Shaaltiel Y. Taliglucerase alfa: An enzyme replacement therapy using plant cell expression technology. Mol Genet Metab. 2014; 112(1):1-8.
He X, Pierce O, Haselhorst T, von Itzstein M, Kolarich D, Packer N H, Gloster T M, Vocadlo D J, Qian Y, Brooks D, Kermode A R “Characterization and downstream mannose phosphorylation of human recombinant α-L-iduronidase produced in Arabidopsis complex glycan-deficient (cgl) seeds” Plant Biotechnol J., 2013, 11(9):1034-1043.
Kakkis E, Lester T, Yang R, Tanaka C, Anand V, Lemontt J, Peinovich M, Passage M. Successful induction of immune tolerance to enzyme replacement therapy in canine mucopolysaccharidosis I. Proc Natl Acad Sci USA. 2004; 101(3):829-834.
Karlin S. and Altschul, S. F. (1990) “Methods for Assessing the Statistical Significance of Molecular Sequence Features by Using General Scoring Schemes” Proc. Natl. Acad. Sci. USA 87:2264-2268.
Karlin S. and Altschul, S. F. (1993) “Applications and Statistics for Multiple High-Scoring Segments in Molecular Sequences” Proc. Natl. Acad. Sci. USA 90:5873-5877.
Kishnani P, Goldenberg P, DeArmey S, Heller J, Benjamin D, Young S, Bali D, Smith S, Li J, Mandel H, Hoeberl D, Rosenberg A. Cross-reactive immunologic material status affects treatment outcomes in Pompe disease infants. Mol Genet Metab. 2010; 99(1):26-33.
Komarova T, Baschieri S, Donini M, Marusic C, Benvenuto E, Dorokhov Y. Transient expression systems for plant-derived biopharmaceuticals. Expert Rev Vaccines. 2010; 9(8):859-76.
Lee A, Sosa A, Montafio A. Development of a less immunogenic protein for enzyme replacement therapy of Morquio syndrome type A disease. Mol Genet Metab. 2015; 114(2):71.
Lewin, B. (1985) Genes II, John Wiley & Sons, Inc., p. 96.
Lovrinovic M and Niemeyer C M “Rapid synthesis of DNA-cysteine conjugates for expressed protein ligation” Biochem Biophys Res Commun., 2005, 335(3):943-948.
Lungwitz U, Breunig M, Blunk T, Göpferich A “Polyethylenimine-based non-viral gene delivery systems” Eur. J. Pharmaceutics Biopharmaceutics, 2005, 60(2):247-266.
Maddaloni M, Cooke C, Wilkinson R, Stout A V, Eng L, Pincus S. Immunological characteristics associated with the protective efficacy of antibodies to ricin. J Immunol. 2004; 172(10):6221-6228.
Maveyraud et al. “Structural basis for sugar recognition, including the tn carcinoma antigen, by the lectin sna-ii from sambucus nigra” Proteins 75 p. 89 (2009).
Medina-Bolivar F, Wright R, Funk V, Sentz D, Barroso L, Wilkins T D, Petri W Jr, Cramer C L “A non-toxic lectin for antigen delivery of plant-based mucosal vaccines” Vaccine, 2003, 21:997-1005.
Montfort et a. “The three-dimensional structure of ricin at 2.8 A” J. Biol Chem. 262 (11), 5398-5403 (1987).
Nayak S, Doerfler P, Porvasnik S, Cloutier D, Khanna R, Valenzano K, Herzog R, Byrne B. Immune responses and hypercoagulation in ERT for pompe disease are mutation and rhGAA dose dependent. PLoS One. 2014; 9(6):1-12.
O'Hara J, Brey R, Mantis N. Comparative efficacy of two leading candidate ricin toxin A subunit vaccines in mice. Clin Vaccine Immunol. 2013; 20(6):789-794.
Olsnes S. The history of ricin, abrin and related toxins. Toxicon. 2004; 44:361-370.
Ou L, Herzog T, Koniar B L, Gunther R, Whitley C. High-dose enzyme replacement therapy in murine Hurler syndrome. Mol Genet Metab. 2014; 111(2):116-122.
Ou, L. Acosta W, Koniarc B, Cooksley R, Cramer C, Radin D, Whitley C. 2016 Enzyme replacement therapy with α-L-iduronidase and lectin RTB fusion protein in treating murine Hurler syndrome. Mol. Genet. Metab. Vol. 117 (2) p. S88.
Pastores G, Shankar S, Szer J, Petakov M, Cox T, Giraldo P, et al. Plant cell-expressed recombinant glucocerebrosidase: Taliglucerase alfa as therapy for Gaucher disease in adults patients previously treated with imiglucerase: 24-month results. Mol Genet Metab. 2013; 108(2):73-4.
Ponder K. Immune response hinders therapy for lysosomal storage diseases. J Clin Invest. 2008; 118(8):2686-2689.
Rozaklis T, Beard H, Hassiotis S, Garcia A, Tonini M, Luck A, et al. Impact of high-dose, chemically modified sulfamidase on pathology in a murine model of MPS IIIA. Exp Neurol. 2011; 230(1):123-130.
Sandvig K, Bergan J, Kavaliauskiene S, Skotland T. Lipid requirements for entry of protein toxins into cells. Prog Lipid Res. 2014; 54C:1-13.
Sandvig K, Pust S, Skotland T, van Deurs B. Clathrin-independent endocytosis: mechanisms and function. Curr Opin Cell Biol. 2011; 23:413-420.
Sandvig K, van Deurs B. Endocytosis and intracellular transport of ricin: recent discoveries. FEBS Letters. 1999; 452(1-2):67-70.
Simmons B, Stahlf P, Russell J. Mannose Receptor-mediated Uptake of Ricin Toxin and Ricin A Chain by Macrophages. J Biol Chem. 1986; 261(17):7912-20.
Turner C, Hopwood J, Brooks D. Enzyme replacement therapy in mucopolysaccharidosis I: altered distribution and targeting of alpha-L-iduronidase in immunized rats. Mol Genet Metab. 2000; 69(4):277-85.
Van Damme et al. “Characterization and molecular cloning of Sambucus nigra agglutinin V (nigrin b), a GalNAc-specific type-2 ribosome-inactivating protein from the bark of elderberry (Sambucus nigra)” Eur. J. Biochem. 237 (2), 505-513 (1996).
Van Damme Els J M, Peumansa W J, Barreb A, Rouge P “Plant Lectins: A Composite of Several Distinct Families of Structurally and Evolutionary Related Proteins with Diverse Biological Roles” Crit. Rev. Plant Sci., 1998, 17(6):575-692.
Van Deurs B, Tonnessen T I, Petersen O, Sandvig K, Olsnes S. Routing of Internalized Ricin and Ricin Conjugates to the Golgi Complex. J Cell Biol. 1986; 102:37-47.
Wang D, Shukla C, Liu X, Schoeb T R, Clarke L, Bedwell D, Keeling K. Characterization of an MPS I-H knock-in mouse that carries a nonsense mutation analogous to the human IDUA-W402X mutation. Mol Genet Metab; 2010; 99(1):62-71.
Wang J, Lozier J, Johnson G, Kirshner S, Verthelyi D, Pariser A, Shores E, Rosenberg A. Neutralizing antibodies to therapeutic enzymes: considerations for testing, prevention and treatment. Nat Biotechnol. 2008; 26(8):901-908.
Whaley K, Hiatt A, Zeitlin L. Emerging antibody products and Nicotiana manufacturing. Hum Vaccines. 2011; 7(3):349-56.
Wraith J, Beck M, Lane R, van der Ploeg A, Shapiro E, Xue Y, Kakkis E, Guffon N. Enzyme replacement therapy in patients who have mucopolysaccharidosis I and are younger than 5 years: results of a multinational study of recombinant human alpha-L-iduronidase (laronidase). Pediatrics. 2007; 120 (1) e37-46
Xu F, Ding E, Liao S X, Migone F, Dai J, Schneider A, Serra D, Chen Y, Amalfitano A. Improved efficacy of gene therapy approaches for Pompe disease using a new, immune-deficient GSD-II mouse model. Gene Ther. 2004; 11(21):1590-1598.

1. A method for delivering an agent into a cell, in the presence of immune components against said agent, the method comprising providing said agent in an environment comprising said cell and comprising immune components directed against said agent, wherein said agent is operatively linked to a lectin-based carrier, and whereby said agent operatively linked to said lectin-based carrier is internalized into said cell by uptake mechanisms directed by the lectin-based carrier.

2. The method according to claim 1, wherein said immune components are immune cells or antibodies or antisera that bind to one or more epitopes of said agent.

3. The method according to claim 1, wherein said immune cells or antibodies or antisera are neutralizing.

4. The method according to claim 1, wherein said lectin-based carrier is a plant lectin, or wherein said lectin-based carrier is a non-toxic carbohydrate binding subunit of a plant toxin.

5. (canceled)

6. The method according to claim 1, wherein said lectin-based carrier is of low immunogenicity or non-immunogenic.

7. The method according to claim 1, wherein said lectin-based carrier is the B-subunit of ricin.

8. The method according to claim 1, wherein said agent is a compound, drug, protein, peptide, antigen, immunogen, or nucleic acid, or wherein said agent is a lysosomal enzyme, such as iduronidase.

9-10. (canceled)

11. The method according to claim 1, wherein said cell is a mammalian cell, or wherein said cell is a human cell.

12. (canceled)

13. A method for treating a disease or condition in a human or animal wherein the human or animal has produced or will produce an immune response against a therapeutic agent that can treat said disease or condition, the method comprising administering to the human or animal an effective amount of said therapeutic agent operatively linked to a lectin-based carrier.

14. The method according to claim 13, wherein said immune response comprises the production of immune cells or antibodies that bind to one or more epitopes of said therapeutic moiety.

15. The method according to claim 14, wherein said immune cells or antibodies are neutralizing.

16. The method according to claim 13, wherein said lectin-based carrier is a plant lectin, or wherein said lectin-based carrier is a non-toxic carbohydrate binding subunit of a plant toxin.

17. (canceled)

18. The method according to claim 13, wherein said lectin-based carrier is the B-subunit of ricin.

19. The method according to claim 13, wherein said lectin-based carrier is of low immunogenicity or non-immunogenic.

20. The method according to claim 13, wherein said agent is a compound, drug, protein, peptide, antigen, immunogen, nucleic acid, or other synthetic or biological molecule that is used in treating or preventing the disease or condition, or wherein said agent is a lysosomal enzyme, such as iduronidase.

21-22. (canceled)

23. The method according to claim 13, wherein said lectin-based carrier is used to simultaneously or subsequently deliver more than one said agent in said human or animal.

24. A method for generating and/or enhancing an immune response in a human or animal against an antigen or immunogen, the method comprising administering to the human or animal an effective amount of said antigen or immunogen, wherein said antigen or immunogen is operatively linked to a lectin-based carrier, and wherein said lectin-based carrier is of low immunogenicity or is not immunogenic and said antigen or immunogen is immunogenic in said human or animal.

25-32. (canceled)

33. The method according to claim 13, wherein said disease or condition is a lysosomal storage disease, or wherein said disease or condition is Hurler disease.

34. (canceled)

35. The method according to claim 13, wherein said lectin-based carrier is used to simultaneously or subsequently deliver two or more different agents to the human or animal.

36. The method according to claim 1, wherein said cell has a lysosomal storage disorder, or wherein said cell is deficient in production of α-L-iduronidase.

37. (canceled)


SEQUENCE LISTING (1 sequence)

SEQ ID NO: 1 - protein, 28 aa, Solanum tuberosum
MASSATTKSFLILFFMILATTSSTCAVD